(19) United States
(12) Patent Application Publication
(10) Pub. No.: US 2024/0062889 A1
(43) Pub. Date: Feb. 22, 2024

(54) MEDICAL INFORMATION PROCESSING DEVICE, MEDICAL INFORMATION PROCESSING METHOD, AND COMPUTER PROGRAM PRODUCT

(71) Applicants: CANON MEDICAL SYSTEMS CORPORATION, Otawara-shi (JP); Canon Kabushiki Kaisha, Tokyo (JP)

(72) Inventors: Koji HIRATA, Kasukabe (JP); Katsuhiko FUJIMOTO, Saitama (JP); Tomoki FUJITO, Nasushiobara (JP)

(73) Assignees: CANON MEDICAL SYSTEMS CORPORATION, Otawara-shi (JP); Canon Kabushiki Kaisha, Tokyo (JP)

(21) Appl. No.: 18/452,151

(22) Filed: Aug. 18, 2023

(30) Foreign Application Priority Data
Aug. 22, 2022 (JP) 2022-131893

(51) Int. Cl. G16H 40/60; G16H 20/10; G16H 20/40

(52) U.S. Cl. CPC G16H 40/60; G16H 20/10; G16H 20/40

(57) ABSTRACT

A medical information processing device according to an embodiment includes processing circuitry configured to acquire information on a cause of damage to a site of a patient, to specify an area where the damage is present in a medical image including the site, and to determine an amount of cells for cell therapy for the damage to the site on the basis of the acquired information on the cause and the specified area where the damage is present.

### CROSS-REFERENCE TO RELATED APPLICATIONS

This application is based upon and claims the benefit of priority from Japanese Patent Application No. 2022-131893, filed on Aug. 22, 2022, the entire contents of which are incorporated herein by reference.

### FIELD

Embodiments described herein relate generally to a medical information processing device, a medical information processing method, and a computer program product.

### BACKGROUND

In the related art, when damage has occurred at a patient's site due to coronary artery occlusion (myocardial infarction), radiation therapy, or anticancer drug therapy, cell therapy, in which cells are introduced to the site of the damage, has been performed in some cases.

Under such circumstances, cell necrosis (damage) caused by anticancer drugs is expanded slowly and in a chain reaction, making it difficult to ascertain the amount of cells to be replenished.

### BRIEF DESCRIPTION OF THE DRAWINGS

FIG. 1 is a diagram illustrating an example of the configuration of a treatment support system according to an embodiment;

FIG. 2 is a diagram illustrating an example of the configuration of a support device according to an embodiment;

FIG. 3 is a flowchart illustrating an example of the workflow of a treatment support according to an embodiment;

FIG. 4 is a flowchart illustrating an example of the flow of a support process performed in the support device according to an embodiment;

FIG. 5 is a flowchart illustrating another example of the workflow of a treatment support according to an embodiment; and

FIG. 6 is a flowchart illustrating another example of the workflow of a treatment support according to an embodiment.

### DETAILED DESCRIPTION

A medical information processing device described in the following embodiment includes processing circuitry configured to acquire information on a cause of damage to a site of a patient, to specify an area where the damage is present in a medical image including the site, and to determine an amount of cells for cell therapy for the damage to the site on the basis of the acquired information on the cause and the specified area where the damage is present.

A medical information processing device, a medical information processing method, a computer program, and a computer program product according to each embodiment are described below with reference to the drawings. In the following description, components having the same or substantially the same functions as those described above with respect to the drawings already illustrated are denoted by the same reference numerals, and redundant description thereof is given only when necessary. Even when the same part is represented, the dimensions and proportions may differ from each other depending on the drawing. For example, from the viewpoint of ensuring the visibility of the drawings, reference numerals are attached only to main components in the description of each drawing, and even components having the same or substantially the same functions may not be attached with reference numerals.

In the related art, when damage has occurred at a patient's site due to coronary artery occlusion (myocardial infarction), radiation therapy, or anticancer drug therapy, cell therapy, in which cells are introduced to the site of the damage, has been performed in some cases.

In the cell therapy, the amount and type of cells to be replenished are determined by specifying an area where damage such as myocardial necrosis has occurred (myocardial necrosis area).

For example, in the case of myocardial necrosis (myocardial infarction) due to coronary artery occlusion, bypass surgery to restart blood flow and supply oxygen can limitedly suppress the exacerbation. In the case of myocardial necrosis due to coronary artery occlusion, when necrosis is suppressed to the extent that it can be saved, a tissue having escaped necrosis compensates for function. In the case of myocardial necrosis due to coronary artery occlusion, the location of occurrence is an area dominated by the occluded coronary artery and is localized. Thus, in the case of myocardial necrosis due to coronary artery occlusion, for example, the amount of cells to be replenished can be ascertained because the necrosis does not expand when bypass surgery is performed. Since the necrosis has locality, the cell type to be replenished is ascertainable.

For example, in the case of myocardial necrosis due to radiation irradiation, since radicals have a short lifespan, exacerbation is transient but limited. In the case of myocardial necrosis due to radiation irradiation, when necrosis is suppressed to the extent that it can be saved, a tissue having escaped necrosis compensates for function. In the case of myocardial necrosis due to radiation irradiation, the location of occurrence is not localized because it follows a dose distribution. In the case of myocardial necrosis due to radiation irradiation, discontinuation is a countermeasure, but as the accuracy of irradiation planning is improved, the number of obstacles is reduced and discontinuation is rare. Thus, in the case of myocardial necrosis due to radiation irradiation, since necrosis is expanded slowly, the amount of cells to be replenished is ascertainable although somewhat difficult. Although necrosis is poorly localized due to radical-induced vascular endothelial cell damage, cell types to be replenished are ascertainable.

Under such circumstances, there were cases such as cell necrosis (damage) caused by anticancer drugs, in which the volume of a myocardial necrosis area is not ascertainable and the amount of cells to be replenished is not ascertainable.

For example, in the case of myocardial necrosis due to anticancer drugs, it has been difficult to stop the spread of necrosis because it is exacerbated by a peroxidative reaction associated with drug metabolism. In the case of myocardial necrosis due to anticancer drugs, there was the problem that the range of necrosis is wide, and functional compensation is not achievable only in tissues having escaped necrosis. In the case of myocardial necrosis due to anticancer drugs, there was the problem that the location of occurrence is not localized because it follows the distribution of the anticancer drugs. In the case of myocardial necrosis due to anticancer drugs, there was the problem that drug withdrawal is a main countermeasure, which interfered with the original cancer treatment.

Therefore, cell necrosis (damage) caused by anticancer drugs is expanded slowly and in a chain reaction, making it difficult to ascertain the amount of cells to be replenished. Furthermore, necrosis was not localized because it was caused by lipid peroxidation associated with drug metabolism, making it difficult to ascertain the cell type to be replenished. On the other hand, for cell necrosis (damage) caused by anticancer drugs, it is required to determine the cell amount and cell type of therapeutic cells early and with a cell amount including a margin for coping with the expansion of necrosis.

In this regard, the present disclosure discloses a treatment support system 1 that can appropriately support cell therapy for site damage.

In the following description, myocardial necrosis due to anticancer drugs is used as an example of site damage. That is, the present embodiment assumes that a site damaged by an anticancer drug is the myocardium. The present embodiment also assume that a drug administered to a patient is an anticancer drug.

### First Embodiment

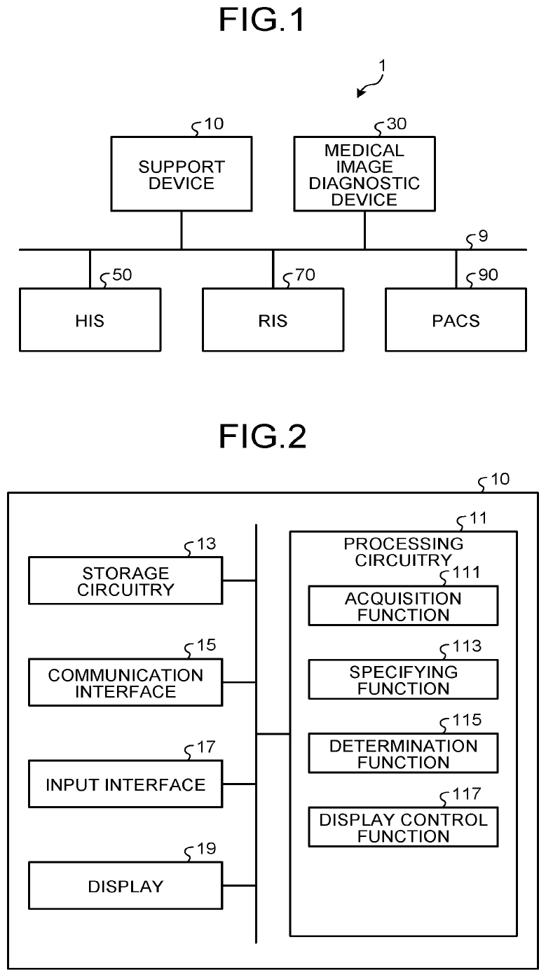

FIG. 1 is a diagram illustrating an example of the configuration of the treatment support system 1 according to an embodiment. As illustrated in FIG. 1, the treatment support system 1 has a support device 10, a medical image diagnostic device 30, a hospital information system (HIS) 50, a radiology information system (RIS) 70, and a medical image management System (picture archiving and communication systems (PACS)) 90. Each device of the treatment support system 1 is installed in, for example, a hospital or the like, and can communicate with other devices through a network 9 such as an in-hospital local area network (LAN). The HIS 50 may be connected to an external network in addition to the hospital LAN. The support device 10 is an example of a medical information processing device.

The HIS 50 is a system that manages information generated in the hospital. The information generated in the hospital includes information such as patient information and test order information. Each record included in the patient information has items such as patient ID, patient name (full name), age (date of birth), sex, height, weight, and blood type. Each record included in the test order information has items such as test ID that can identify a test, patient ID, information indicating inpatient or outpatient status, test code, clinical department, test type, test site, and scheduled test date.

The test ID is issued when the test order information is input and is an identifier for uniquely specifying test order information within one hospital, for example. The patient ID is assigned to each patient and is an identifier for uniquely specifying a patient within one hospital, for example. The test code is an identifier for uniquely specifying a test defined within one hospital, for example. The clinical department, for example, indicates the specialty category of medical treatment in medicine. Specifically, the clinical department includes internal medicine and surgery. The test type indicates an examination using medical images. For example, the test type includes an X-ray test, a computed tomography (CT) test, a magnetic resonance imaging (MRI) test, and the like. The test site includes brain, kidney, lung, liver, and the like.

For example, when the test order information is input by a test ordering physician, the HIS 50 transmits, to the RIS, the input test order information and patient information specified by the test order information. In this case, the HIS 50 also transmits the patient information to the PACS.

The RIS 70 is a system that manages test reservation information on radiographic test work. For example, the RIS 70 receives the test order information transmitted from the HIS 50, adds various setting information to the received test order information, accumulates the information, and manages the accumulated information as test reservation information. Specifically, when the patient information and the test order information transmitted from the HIS 50 are received, the RIS 70 generates test reservation information necessary for operating the medical image diagnostic device 30 on the basis of the received patient information and test order information. The test reservation information includes, for example, necessary information for performing a test such as test ID, patient ID, test type, and test site. The RIS 70 transmits the generated test reservation information to the medical image diagnostic device 30.

The medical image diagnostic device 30 is a device that generates medical image data on the basis of data collected from a subject. The medical image diagnostic device 30 that can be appropriately used includes various medical image diagnostic devices such as an X-ray diagnostic device, an X-ray computed tomography (CT) device, a magnetic resonance imaging (MRI) device, an ultrasonic diagnostic device, a single photon emission computed tomography (SPECT) device, a positron emission computed tomography (PET) device, a SPECT-CT device in which the SPECT device and the X-ray CT device are integrated, and a PET-CT device in which the PET device and the X-ray CT device are integrated.

The medical image diagnostic device 30 performs a test on the basis of the test reservation information transmitted from the RIS 70, for example. The medical image diagnostic device 30 generates test execution information indicating the execution of the test, and transmits the test execution information to the RIS 70. In this case, the RIS 70 receives the test execution information from the medical image diagnostic device 30, and outputs the received test execution information to the HIS 50 or the like as the latest test execution information. For example, the HIS 50 receives the latest test execution information, and manages the received test execution information. The test execution information includes test reservation information such as test ID, patient ID, test type, and test site, and test execution date and time.

The medical image diagnostic device 30 converts the generated medical image data into a format conforming to, for example, a digital imaging and communication in medicine (DICOM) standard. That is, the medical image diagnostic device 30 generates, as supplementary information, medical image data to which a DICOM tag is added.

The supplementary information includes, for example, patient ID, test ID, device ID, image series ID, and the like, and is standardized according to the DICOM standard. The device ID is information for identifying the medical image diagnostic device 30. The image series ID is information for identifying one-time imaging by the medical image diagnostic device 30, and includes, for example, a site of a subject (patient) imaged, image generation time, slice thickness, slice position, and the like. For example, by performing a CT test or an MRI test, a tomographic image at each of a plurality of slice positions is obtained as medical image data.

The medical image diagnostic device 30 transmits the generated medical image data to the PACS 90. The PACS 90 is a system that manages various medical image data.

The PACS 90, for example, receives the patient information transmitted from the HIS 50, and manages the received patient information. The PACS 90 includes storage circuitry for managing the patient information. The PACS 90 receives, for example, the medical image data transmitted from the medical image diagnostic device 30, and stores the received medical image data in its own storage circuitry in association with the patient information. The supplementary information such as patient ID, test ID, device ID, and image series ID is added to the medical image data stored in the PACS 90. Therefore, an operator can acquire necessary patient information from the PACS 90 by performing a search using the patient ID or the like. An operator can also acquire necessary medical image data from the PACS 90 by performing a search using the patient ID, the test ID, the device ID, or the image series ID.

The HIS 50 receives, for example, an electronic medical record prepared by a clinician who is a test ordering physician, and the test execution information corresponding to the electronic medical record, and stores the received electronic medical record and test execution information in its own storage circuitry by associating them. As described above, since the test execution information includes test ID, patient ID, test type, test site, test execution date and time, and the like, an operator can acquire necessary electronic medical record from the HIS 50 by performing a search using the patient ID, the test ID, and the like. In the present embodiment, the electronic medical record is stored in the storage circuitry of the HIS 50, but may be stored in storage circuitry of another device in the treatment support system 1 as long as it can be retrieved by ID.

The RIS 70 also receives, for example, an interpretation report produced in response to input from a radiologist and test execution information corresponding to the interpretation report, and stores the received interpretation report and test execution information in its own storage circuitry by associating them. As described above, since the test execution information includes test ID, patient ID, test type, test site, test execution date and time, and the like, an operator can acquire a necessary interpretation report from the RIS 70 by performing a search using the patient ID, the test ID, and the like. In the present embodiment, the interpretation report is stored in the storage circuitry of the RIS 70, but may be stored in storage circuitry of another device in the treatment support system 1 as long as it can be retrieved by ID.

The support device 10 performs a support process. The support device 10 acquires various medical treatment data from the medical image diagnostic device 30, the HIS 50, the RIS 70, and the PACS 90 via the network 9, and performs various types of information processing by using the acquired medical treatment data. For example, the support device 10 is implemented by a computer such as a workstation having a processor and a memory such as ROM and RAM as hardware resources. For example, an integrated viewer is installed in the support device 10. The integrated viewer is an application that presents medical information to a user in an integrated manner. The integrated viewer may adopt any implementation form, such as a web application, a fat client application, or a thin client application.

The medical treatment data is information indicating medical treatment records obtained by a medical worker regarding patient's physical conditions, medical conditions, treatment, and the like in the course of medical treatment. The medical treatment data includes, for example, data acquired in various environments such as devices from different manufacturers, different versions of devices, and different settings even for the same device. The medical treatment data is not limited to objective data such as numerical values, but may also include non-numerical data, for example, subjective data represented by characters. The medical treatment data includes, for example, test history information, image information, electrocardiogram information, vital sign information, medication history information, report information, medical record description information, nursing record information, referral letters, discharge summaries, and the like. The test history information is, for example, information representing the history of test results acquired as a result of specimen tests, bacteriological tests, and the like performed on patients. The image information is, for example, information representing the location of medical images acquired by imaging a patient. The image information includes, for example, information representing the location of a medical image file generated by the medical image diagnostic device as a result of a test. The electrocardiogram information is, for example, information on an electrocardiogram waveform measured from a patient. The vital sign information, for example, is basic information on a patient's life. The vital sign information includes, for example, pulse rate, respiratory rate, body temperature, blood pressure, level of consciousness, and the like. The medication history information is, for example, information indicating the history of the amount of medication administered to a patient. The report information is, for example, information that summarizes patient's conditions and disease after a radiologist interprets medical images such as X-ray images, CT images, MRI images, and ultrasound images in response to a test request from a medical doctor in a clinical department. The report information includes, for example, interpretation report information representing an interpretation report produced by a radiologist by referring to a medical image file stored in the PACS. The report information includes, for example, information representing the patient ID, patient name, and date of birth of a patient corresponding to the medical image file to be interpreted. The medical record description information is, for example, information input to an electronic medical record by a medical doctor or the like. The medical record description information includes, for example, medical treatment records during hospitalization, the patient's medical history and drug prescription history, and the like. The nursing record information is, for example, information input to the electronic medical record by a nurse or the like. The nursing record information includes nursing records during hospitalization. The nursing record information may include meal records during hospitalization. The medical treatment data may further include information on accounting.

The treatment support system 1 may have a vendor neutral archive (VNA) system instead of the HIS 50, the RIS 70, and the PACS 90. The VNA system is an integrated archive system that centrally manages PACS 90 manufactured by another manufacturer and various medical treatment data managed by each clinical department system (the HIS 50 and the RIS 70). The VNA system is communicably connected to, for example, the HIS 50, the RIS 70, and the PACS 90 via an in-hospital network such as LAN. Various information managed and stored by the VNA system is not necessarily acquired from systems manufactured by different manufacturers, but may be acquired from systems manufactured by a single manufacturer.

FIG. 2 is a diagram illustrating an example of the configuration of the support device 10 according to the embodiment. As illustrated in FIG. 2, the support device 10 has processing circuitry 11, storage circuitry 13, a communication interface 15, an input interface 17, and a display 19. The processing circuitry 11, the storage circuitry 13, the communication interface 15, the input interface 17, and the display 19 are communicatively connected to one another via a bus or the like.

The storage circuitry 13 stores various data. For example, the storage circuitry 13 stores image data and medical treatment data received from the medical image diagnostic device 30, the HIS 50, the RIS 70, and the PACS 90. For example, the storage circuitry 13 stores computer programs for implementing a support process to be described below. For example, the storage circuitry 13 is implemented by, for example, a semiconductor memory element such as a random access memory (RAM) and a flash memory, a hard disk, an optical disk, or the like. A storage area of the storage circuitry 13 may be in the support device 10 or in an external storage device connected by a network or the like. The storage circuitry 13 is an example of a storage unit.

The communication interface 15 controls transmission and communication of various data among the medical image diagnostic device 30, the HIS 50, the RIS 70, and the PACS 90. For example, the communication interface 15 receives image data and medical treatment data from the medical image diagnostic device 30, the HIS 50, the RIS 70, and the PACS 90, and outputs the received data to the processing circuitry 11. The communication interface is implemented, for example, by a network card, a network adapter, a network interface controller (NIC), or the like.

The input interface 17 receives various input operations from an operator, converts the received input operations into electrical signals, and outputs the electrical signals to the processing circuitry 11. The input interface 17, for example, receives various input operations from an operator on various operation screens related to the support process. As an example, the input interface 17 receives input operations related to the selection of therapeutic techniques by an operator. The input interface 17 is an example of an input unit.

Examples of the input interface 17 that can be appropriately used include a mouse, a keyboard, a trackball, a switch, a button, a joystick, a touch pad, and a touch panel display. In the present embodiment, the input interface 17 is not limited to those having physical operation parts. For example, the input interface 17 also includes electrical signal processing circuitry that receives an electrical signal corresponding to an input operation from an external input device provided separately from the device and outputs the electrical signal to the processing circuitry 11. The input interface 17 may also be configured as a tablet terminal or the like capable of wirelessly communicating with the main body of the support device 10.

The display 19 displays various information. The display 19 outputs, for example, a graphical user interface (GUI) or the like generated by the processing circuitry 11 to receive various operations from an operator. The GUI for receiving various operations from an operator includes various operation screens related to the support process. For example, the display 19 outputs a display screen related to the support process and generated by the processing circuitry 11. As an example, the display 19 outputs a display screen including a list of therapeutic techniques that contribute to cell therapy, and a display screen including cell types and amount of cells for the therapeutic techniques. As the display 19, various arbitrary displays can be appropriately used. Examples of the display 19 that can be used include a liquid crystal display (LCD), a cathode ray tube (CRT) display, an organic electroluminescence display (OELD), and a plasma display. The display 19 is an example of a display.

The display 19 may be of a desktop type, or may be configured as a tablet device capable of wirelessly communicating with the main body of the support device 10. One or two or more projectors may also be used as the display 19.

The processing circuitry 11 controls the entire operation of the support device 10. The processing circuitry 11 has a processor and a memory such as ROM and RAM as hardware resources. The processing circuitry 11 executes an acquisition function 111, a specifying function 113, a determination function 115, a display control function 117, and the like by the processor executing a computer program loaded to the memory.

The processing circuitry 11 is an example of a processing unit. The processing circuitry 11 that implements the acquisition function 111 is an example of an acquisition unit. The processing circuitry 11 that implements the specifying function 113 is an example of a specifying unit. The processing circuitry 11 that implements the determination function 115 is an example of a determination unit. The processing circuitry 11 that implements the display control function 117 is an example of a display control unit.

In the acquisition function 111, the processing circuitry 11 acquires medical image data and medical treatment data from the medical image diagnostic device 30, the HIS 50, the RIS 70, or the PACS 90 via the network 9. The processing circuitry 11 further acquires the results of operator inputs received by the input interface 17.

As an example, in the acquisition function 111, the processing circuitry 11 acquires medical image data depicting the heart. The heart is an example of a patient's site including an area where anticancer drug damage is present.

As an example, in the acquisition function 111, the processing circuitry 11 acquires medical treatment data including information on the cause of damage to a patient's site. The information on the cause of damage to the patient's site includes information on the administration of an anticancer drug (medicine) to the patient. The information on the administration of the medicine to the patient includes information on the type of the anticancer drug. The information on the administration of the medicine to the patient includes information on the dosage of the anticancer drug. As an example, the information on the dosage of the anticancer drug includes information on the total dosage of the anticancer drug at a specific time point when the specifying function 113 specifies an area where the damage is present.

In the specifying function 113, the processing circuitry 11 specifies an area where damage is present in a medical image including the myocardium. Specifically, the processing circuitry 11 identifies the necrotic structure of the myocardium, and calculates the size (necrotic volume) of the specified area where the damage is present. The processing circuitry 11 may specify the area where the damage is present on the basis of the output of the input interface 17 in response to an operator's operation input such as a doctor, or may specify the area where the damage is present on the basis of the results of image processing on the medical image data including the myocardium.

In the determination function 115, the processing circuitry 11 determines the amount of cells for cell therapy for myocardial damage on the basis of the information on the cause acquired by the acquisition function 111 and the area where the damage is present specified by the specifying function 113. On the basis of a tissue structure identified by the specifying function 113, the processing circuitry 11 determines a cell type for the cell therapy. Specifically, on the basis of the total dosage of the anticancer drug at the specific time point when the specifying function 113 specifies the area where the damage is present and the size (necrotic volume) of the specified area where the damage is present, the processing circuitry 11 predicts an area where damage is present at the time point of disappearance when the occurrence of damage caused by the anticancer drug converges. On the basis of the size of the predicted area where the damage is present (predicted necrotic volume), the processing circuitry 11 also determines the amount of cells for the cell therapy.

In the display control function 117, the processing circuitry 11 displays, on the display 19, a display screen including a list of the therapeutic techniques that contribute to the cell therapy. Specifically, the processing circuitry 11 determines the display order of the therapeutic techniques on the basis of information on the patient's surgery and the degree of invasiveness of the therapeutic technique. The processing circuitry 11 further displays a display screen including a list of therapeutic techniques that contribute to the cell therapy in the determined display order. The processing circuitry 11 may display a display screen including each therapeutic technique by switching the display of the therapeutic techniques that contribute to the cell therapy on the display screen in the determined display order, for example, according to the output of the input interface 17 in response to an operator's operation input.

In the display control function 117, the processing circuitry 11 displays, on the display 19, a display screen including cell types and amount of cells for a selected treatment technique.

The support device 10 according to the embodiment may also be mounted on a culturing device for culturing cells. Alternatively, the support device 10 may be configured to output calculated cell type and amount of cells for the cell therapy to the culturing device.

Each of the functions 111, 113, 115, and 117 is not limited to being implemented by single processing circuitry. The processing circuitry 11 may be configured by combining a plurality of independent processors, and respective processors may implement the functions 111, 113, 115, and 117 by executing computer programs. The functions 111, 113, 115, and 117 may be implemented by being appropriately distributed or integrated into single processing circuitry or a plurality of pieces of processing circuitry.

Although the support device 10 that executes a plurality of functions with a single computer is exemplified, the present disclosure is not limited thereto. The plurality of functions of the support device 10 may be performed by separate computers. For example, the functions of the processing circuitry 11, such as the acquisition function 111, the specifying function 113, and the determination function 115, may be configured to be distributed to and executed by at least two computers.

The support process by the treatment support system 1 according to the embodiment is described below with reference to the drawings.

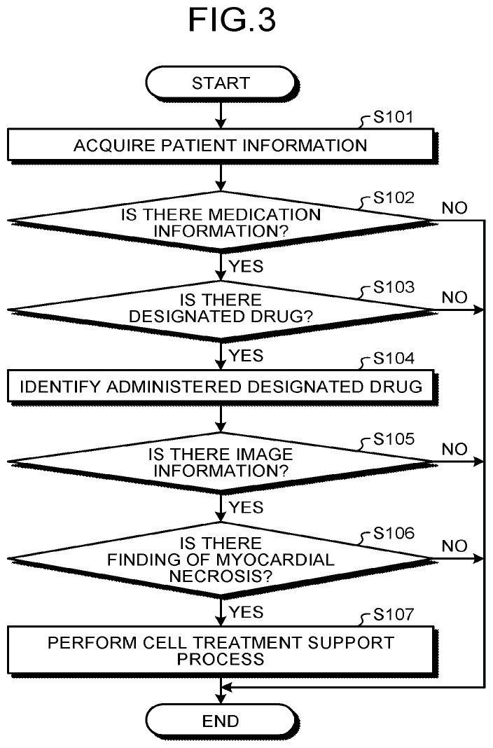

FIG. 3 is a flowchart illustrating an example of the workflow of a treatment support according to the embodiment.

First, information on a patient (medical treatment data) is acquired (S101).

As an example, the acquisition function 111 acquires the medical treatment data.

When the medical treatment data includes medication information (medication history information) (Yes at S102) and includes a designated drug related to the myocardium (Yes at S103), the designated drug administered to the patient is identified (S104).

As an example, the specifying function 113 determines whether the medication history information is registered in the medical treatment data. As an example, the specifying function 113 determines whether the medication history information includes the designated drug related to the myocardium. As an example, the specifying function 113 identifies the designated drug related to the myocardium in the medication history information. These determination and identification may be made by an operator such as a doctor who confirms the medical treatment data displayed on the display 19 or the like.

The designated drug is assumed to be a drug that generates reactive oxygen species in cells in the process of drug metabolism. When an anticancer drug that generates reactive oxygen species in a metabolic process is administered, it continues to induce oxidative stress until the time point of disappearance when the distribution concentration reaches zero, causing cell damage in a dose-dependent manner. That is, the administration of the designated drug may cause myocardial damage (injury). Specifically, the reactive oxygen species have effects on intracellular signaling or its triggering, DNA inhibition, and RNA inhibition. Therefore, while the designated drug has medicinal effects on target cells such as cancer cells, it has side effects such as DNA inhibition, RNA inhibition, and impaired biomembrane material peroxidation on off-target cells such as myocardial cells. The designated drug is determined in advance and stored in the storage circuitry 13 or the like, for example. Alternatively, information indicating the designated drug may be attached to the medical treatment data.

Examples of the designated drug may include anthracycline anticancer drugs such as doxorubicin, daunorubicin, mitoxantrone, and epirubicin. Examples of the designated drug may include antibiotic anticancer drugs such as mitomycin C and bleomycin. Examples of the designated drug may include alkylating drugs such as cyclophosphamide and cisplatin. Examples of the designated drug may include antimetabolites such as fluorouracil and methotrexate. Examples of the designated drug may include plant alkaloids such as vincristine and etoposide. These designated drugs are examples, and various drugs that may cause damage to a target patient's site may be set as the designated drugs.

When the medical treatment data does not include the medication information (medication history information) (No at S102) or when the medical treatment data does not include the designated drug related to the myocardium (No at S103), the flow in FIG. 3 ends.

After the designated drug administered to the patient has been identified, when the medical treatment data includes image information (Yes at S105) and a finding of myocardial necrosis is present (Yes at S106), a cell treatment support process to be described below is performed (S107). The presence of the image information means the presence of image data depicting the target myocardium (heart). The presence of the finding of the myocardial necrosis means, for example, that report information of the medical treatment data includes findings suggestive of the myocardial necrosis.

As an example, the image information and the findings of myocardial necrosis are based on image data obtained by delayed contrast-enhanced MRI techniques. As another example, the image data may be image data obtained by a photon counting CT (PCCT) method.

As an example, the specifying function 113 determines whether the image information is registered in the medical treatment data. As an example, the specifying function 113 determines whether the report information includes findings suggestive of the myocardial necrosis. These determinations may be made by an operator such as a doctor who confirms the medical treatment data displayed on the display 19 or the like.

When the medical treatment data does not include the image information (No at S105) and the finding of the myocardial necrosis is not present (No at S106), the flow in FIG. 3 ends.

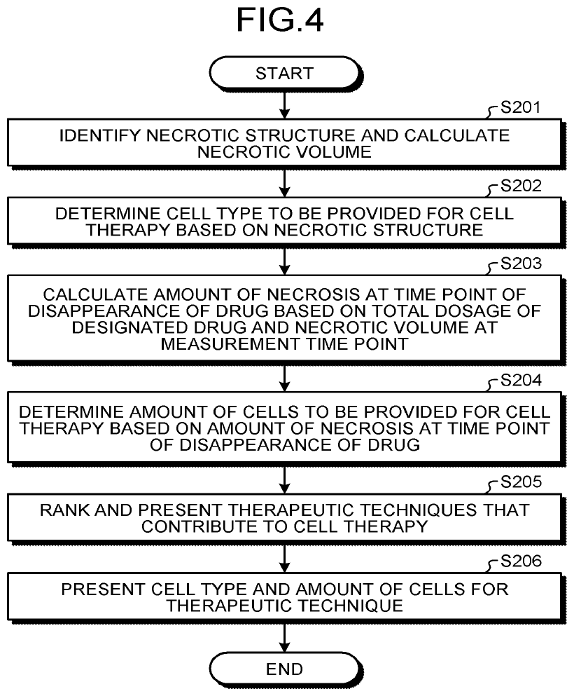

FIG. 4 is a flowchart illustrating an example of the flow of the support process performed in the support device 10 according to the embodiment.

The specifying function 113 identifies the necrotic structure of the myocardium on the basis of the medical image data acquired by the acquisition function 111, specifies an area of existing damage, and calculates the size of the area, that is, the necrotic volume of the myocardium at the specific time point (S201).

The determination function 115 determines a cell type to be provided for cell therapy on the basis of the necrotic structure identified by the specifying function 113 (S202). On the basis of the total dosage of the designated drug at the specific time point and the necrotic volume of the myocardium at the specific time point (measurement time point), the determination function 115 calculates the amount of myocardial necrosis at the time point of disappearance when the occurrence of damage caused by the anticancer drug converges (S203). On the basis of the calculated amount of myocardial necrosis at the time point of disappearance, the determination function 115 determines the amount of cells to be provided for the cell therapy (S204). Assume that a relational formula or a table showing the relationship between the amount of myocardial necrosis and the amount of cells and changes over time (rate of decrease) of the concentration of the designated drug are determined in advance and stored in the storage circuitry 13 or the like.

The display control function 117 ranks and presents therapeutic techniques that contribute to the cell therapy (S205). Examples of the therapeutic techniques that contribute to the cell therapy include cell transplantation (myocardium), cell transplantation (myoblasts), myocardial induction, stem cell transplantation (mesenchymal stem cells), and stem cell transplantation (muse cells) in descending order of the degree of invasiveness to patients.

The cell transplantation (myocardium) is a therapeutic technique in which sheets or clumps of differentiated cells derived from iPS cells are transplanted. A route of cell introduction of the cell transplantation (myocardium) is, for example, a thoracotomy surgery. The cell transplantation (myoblast) is a therapeutic technique in which sheets of skeletal myoblasts are attached to a surface, with the expectation of a paracrine effect. A route of cell introduction of the cell transplantation (myoblast) is, for example, a thoracotomy surgery. The myocardial induction is a therapeutic technique in which gene clusters are introduced into fibroblasts for direct reprogramming. A route of introduction of the gene clusters of the myocardial induction is, for example, cardiac catheterization. The stem cell transplantation (mesenchymal stem cell) is a therapeutic technique in which mesenchymal stem cells are administered with the expectation of a paracrine effect at a recipient site. A route of cell introduction of the stem cell transplantation (mesenchymal stem cells) is, for example, intravenous injection. The stem cell transplantation (muse cells) is a therapeutic technique in which muse cells are administered, with the expectation of differentiation at the recipient site. A route of cell introduction of the stem cell transplantation (muse cells) is, for example, intravenous injection. These treatment techniques are examples, and various cell therapies that compensate for damage to a target patient's site can be set as display targets.

As an example, the display control function 117 displays therapeutic techniques that contribute to the cell therapy in a predetermined order. For example, assume that the display order of the therapeutic techniques is set for each hospital or each doctor and stored in the storage circuitry 13 or the like. For example, higher rankings may be given to proven treatment techniques on the basis of the clinical reports of a doctor who determines treatment plans.

For example, on the basis of the output of the input interface 17 in response to an operator's operation input such as a doctor, the display control function 117 presents the cell type and amount of cells to be produced for a therapeutic technique selected by the operator (S206).

In this way, the treatment support according to the present embodiment determines the amount of cells for cell therapy for myocardial necrosis on the basis of information on a cause regarding the administration of an anticancer drug that contribute to myocardial necrosis and an area with necrosis specified in a medical image. According to this configuration, the amount and type of cells to be replenished can be appropriately ascertained for damage to a site where a necrotic area is expanded slowly and in a chain reaction, such as myocardial necrosis due to an anticancer drug, and where the localization of the necrotic area is low. In other words, the treatment support according to the present embodiment can appropriately support cell therapy for site damage.

### Second Embodiment

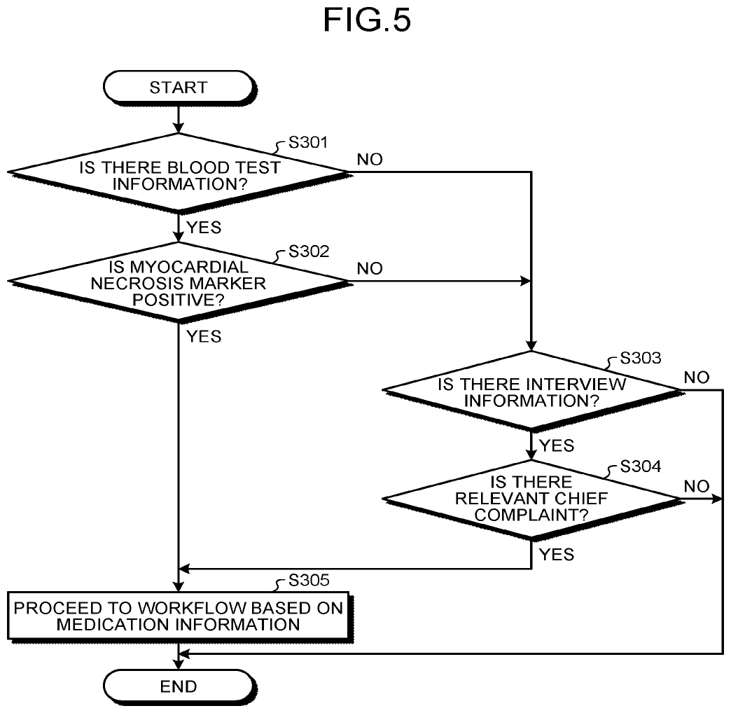

The treatment support according to the embodiment described above can be implemented when supplementary information strongly suggestive of myocardial necrosis is obtained. FIG. 5 is a flowchart illustrating another example of the workflow of a treatment support according to an embodiment.

When the test history information of the medical treatment data includes blood test information indicating blood test results (Yes at S301) and a myocardial necrosis marker is positive in the blood test information (Yes at S302), the flow in FIG. 5 proceeds to the workflow in FIG. 3 based on medication information (S305). The blood test information is an example of supplementary information.

As an example, the myocardial necrosis marker is a marker that increases in blood with the occurrence of myocardial necrosis. The myocardial necrosis marker is, for example, troponin T (TnT), but markers such as CK/CK-MB (creatinine kinase), troponin I (TnI), heart-type fatty acid binding protein (H-FABP), and cardiac myosin light chain I can be appropriately used. For example, since the time from myocardial necrosis to the rise of the marker is different, the myocardial necrosis marker may be selected according to the time from the start of drug administration.

Liquid biopsy data may also be used as the myocardial necrosis marker. As an example, microRNAs (miRNAs) such as miR-1, miR-21, miR-30, miR-126, miR-133, miR-195, miR-208a, miR-208b, miR-328, miR-378, miR-499, miR-1291, and miR-let-7b can be appropriately used.

As an example, the acquisition function 111 acquires medical treatment data including test history information. As an example, the specifying function 113 determines whether the blood test information is registered in the test history information. As an example, the specifying function 113 determines whether the myocardial necrosis marker is positive in the blood test information. As another example, the specifying function 113 determines whether the value of the myocardial necrosis marker in the blood test information is equal to or greater than a predetermined threshold. The threshold value is assumed to be determined in advance and stored in the storage circuitry 13 or the like, for example. These determinations may be made by an operator such as a doctor who confirms the medical treatment data displayed on the display 19 or the like.

When the test history information does not include the blood test information in (No at S301) or the myocardial necrosis marker is negative in the blood test information (No at S302), the flow in FIG. 5 proceeds to S303.

When the medical record description information of the medical treatment data includes interview information at the time of medical treatment (Yes at S303) and the interview information includes a relevant chief complaint indicating a patient's chief complaint related to myocardial necrosis (Yes at S304), the flow in FIG. 5 proceeds to the workflow in FIG. 3 based on the medication information (S305). The interview information is an example of supplementary information.

As an example, the relevant chief complaint includes a chief complaint such as chest pain, chest tightness, shortness of breath, upper abdominal pain, right shoulder pain, and dental pain.

As an example, the acquisition function 111 acquires medical treatment data including the medical record description information. As an example, the specifying function 113 determines whether the interview information is registered in the medical record description information. As an example, the specifying function 113 determines whether the interview information includes a relevant chief complaint indicating a patient's chief complaint related to myocardial necrosis. These determinations may be made by an operator such as a doctor who confirms the medical treatment data displayed on the display 19 or the like.

When the medical record description information does not include the interview information (No at S303) or the interview information does not include the relevant chief complaint (No at S304), the flow in FIG. 5 ends.

The workflow in FIG. 5 may be a flow in which the workflow (S303, S304, and S305) based on the interview information is performed prior to the workflow (S301, S302, and S305) based on the blood test information. The workflow in FIG. 5 may not include either the workflow based on the blood test information or the workflow based on the interview information. The workflow in FIG. 5 may be a flow in which one of the workflow based on the blood test information and the workflow based on the interview information is promoted by the other one thereof.

In this way, in the treatment support according to the present embodiment, the workflow in FIG. 3 based on the medication information is promoted on the basis of at least one of the blood test information and the interview information. In other words, in the treatment support according to the present embodiment, the workflow in FIG. 3 based on the medication information is triggered by a workflow based on at least one of the blood test information and the interview information. According to this configuration, the workflow in FIG. 3 based on the medication information can be performed at the timing when myocardial necrosis is suspected, so that cell therapy for the myocardial necrosis can be appropriately supported.

### Third Embodiment

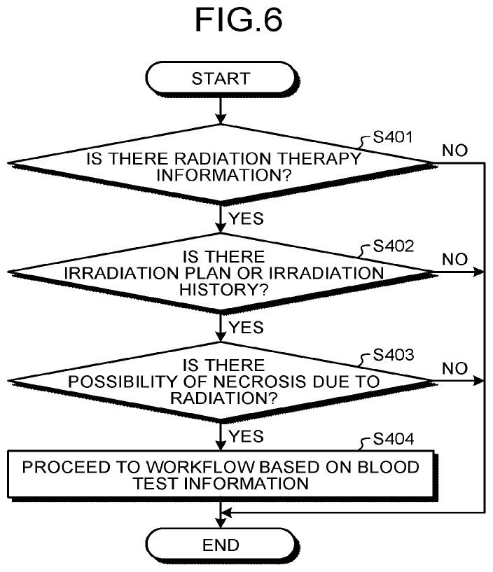

Supplementary information strongly suggestive of myocardial necrosis is not limited to at least one of the blood test and the interview information described above, but may also use radiation therapy information. FIG. 6 is a flowchart illustrating another example of the workflow of a treatment support according to an embodiment.

When the medical treatment data includes information on radiation therapy for a patient (radiation therapy information) (Yes at S401), the radiation therapy information includes an irradiation plan or an irradiation history (Yes at S402), and there is a possibility of necrosis due to radiation irradiation in the irradiation plan or the irradiation history (Yes at S403), the flow in FIG. 6 proceeds to the workflow of FIG. 5 based on at least one of the blood test information and the interview information (S404). The radiation therapy information is an example of supplementary information. The possibility of necrosis due to the radiation irradiation is an example of a predetermined condition for the irradiation plan or the irradiation history.

As an example, the acquisition function 111 acquires medical treatment data including the radiation therapy information. As an example, the specifying function 113 determines whether an irradiation plan or an irradiation history of radiation to the myocardium is registered in the radiation therapy information. As an example, on the basis of dose information and irradiation intervals indicated by the irradiation plan or the irradiation history of the radiation to the myocardium, the specifying function 113 determines whether there is a possibility of myocardial necrosis due to radiation irradiation. Assume that a database showing the relationship between radiation dose and tissue damage is stored in the storage circuitry 13 or the like, for example. These determinations may be made by an operator such as a doctor who confirms the medical treatment data displayed on the display 19 or the like.

The workflow in FIG. 6 based on the radiation therapy information may be a flow that proceeds to the workflow in FIG. 3 based on the medication information, instead of the workflow in FIG. 5 based on at least one of the blood test information and the interview information. The workflow in FIG. 6 based on the radiation therapy information may be a flow that is prompted by the workflow in FIG. 5, that is, a flow that is performed after the workflow in FIG. 5 based on at least one of the blood test information and the interview information. The workflow in FIG. 6 based on the radiation therapy information may be a flow that is performed between the workflow in FIG. 5 based on the blood test information and the workflow in FIG. 5 based on the interview information, is prompted by any one of the workflow based on the blood test information and the workflow based on the interview information, and is prompted by the other one thereof.

In this way, in the treatment support according to the present embodiment, the workflow in FIG. 3 based on the medication information is promoted on the basis of the radiation therapy information. Specifically, in the treatment support, the workflow in FIG. 3 based on the medication information is performed when there is a possibility of necrosis due to radiation irradiation. According to this configuration, the workflow in FIG. 3 based on the medication information can be performed at the timing when myocardial necrosis is assumed, so that cell therapy for the myocardial necrosis can be appropriately supported.

### Fourth Embodiment

In the treatment support according to each of the embodiments described above, examples of the cause information on the administration of an anticancer drug that contributes to myocardial necrosis are provided, but the present disclosure is not limited thereto. Information on radiation therapy (radiation therapy information) can also be used as the cause information.

As an example, in the acquisition function 111, the processing circuitry 11 acquires medical treatment data including information on the cause of damage to a patient's site. The information on the cause of the damage to the patient's site includes a plan or a history of the dose to the site in the radiation therapy. Such dose plan or history includes dose information and irradiation intervals.

In the specifying function 113, the processing circuitry 11 calculates the size (necrotic volume or necrosis amount) of a myocardial necrosis area due to radiation irradiation on the basis of dose information and irradiation intervals indicated by an irradiation plan or an irradiation history of radiation to the myocardium. The processing circuitry 11 may also estimate a necrotic structure on the basis of, for example, a necrotic ratio to dose for each cell type. Assume that a database showing the relationship between radiation dose and tissue damage is stored in the storage circuitry 13 or the like, for example.

As an example, on the basis of the size (necrotic volume or necrosis amount) of the myocardial necrosis area due to the radiation irradiation, the processing circuitry 11 in the determination function 115 corrects the size of an area when damage exists at the time point of disappearance predicted based on cause information on the administration of an anticancer drug to a patient. For example, the processing circuitry 11 determines the amount of cells for cell therapy on the basis of the larger one of the size of the area where damage exists at the time point of disappearance and the size of an area where damage exists at the time point after radiation irradiation according to an irradiation plan or an irradiation history. The processing circuitry 11 may also correct an identified necrotic structure on the basis of an estimated necrotic structure.

As another example, on the basis of the size (necrotic volume or necrosis amount) of the myocardial necrosis area due to the radiation irradiation, the processing circuitry 11 in the determination function 115 corrects the amount of cells for the cell therapy calculated based on the cause information on the administration of the anticancer drug to the patient. For example, the processing circuitry 11 determines, as the amount of cells for the cell therapy, the larger one of the amount of cells based on the size of the area where damage exists at the time point of disappearance and the amount of cells based on the size of the area where damage exists at the time point after the radiation irradiation. The processing circuitry 11 may also correct a cell type determined on the basis of the identified necrotic structure by a cell type determined on the basis of the estimated necrotic structure.

As another example, on the basis of the size (necrotic volume or necrosis amount) of a myocardial necrosis area due to radiation irradiation according to an irradiation plan or an irradiation history for the time point later than the specific time point at which the specifying function 113 specifies the area where damage is present, and the size (necrotic volume) of the specified area where damage is present, the processing circuitry 11 in the determination function 115 predicts an area where damage is present at the time point after radiation is applied to the myocardium according to the irradiation plan or the irradiation history. The processing circuitry 11 further determines the amount of cells for the cell therapy on the basis of the size (predicted necrotic volume or necrosis amount) of the predicted area where damage is present. In this way, for example, when no anticancer drug is being administered, cause information on the administration of a drug may not be used. The processing circuitry 11 may also determine a cell type on the basis of an estimated necrotic structure.

In this way, in the treatment support according to the present embodiment, the amount of cells for cell therapy is determined on the basis of radiation therapy information (cause information). According to this configuration, even when radiation therapy that contributes to myocardial necrosis is used in combination with medication therapy, the amount and type of cells to be replenished can be appropriately calculated, so that cell therapy for myocardial necrosis can be appropriately supported.

### Fifth Embodiment

In the treatment support according to each of the embodiments described above, the amount of cells and the cell type for the cell therapy may be further calculated on the basis of the viability rate of cultured cells introduced into a target site.

In the acquisition function 111, the processing circuitry 11 acquires information on the viability rate of cells to be provided for the cell therapy to the site. The processing circuitry 11 may acquire the viability rate from an external device of the support device 10, such as a culture device (not illustrated) or a management device that manages the culture, or may acquire the viability rate on the basis of the output of the input interface 17 in response to an operator's operation input.

In the determination function 115, the processing circuitry 11 corrects the amount of cells calculated on the basis of the cause information according to the viability rate. For example, as the viability rate decreases, the processing circuitry 11 increases the amount of cells calculated on the basis of the cause information.

For example, the processing circuitry 11 corrects the acquired viability rate by using a relational formula or a table showing the relationship between a viability rate and a correction ratio determined in advance and stored in the storage circuitry 13 or the like. As an example, when the viability rate is 3%, the processing circuitry 11 corrects the amount of cells by a factor of 33.

The relational formula or the table may indicate the relationship between the viability rate and the amount of correction. The relational formula or the table can be prepared for each cell type.

When the viability rate is expected to be improved by changing a cell type to be introduced, for example, a cell type may be determined according to the viability rate, for example, by adding a cell type when there is a viability rate equal to or less than a predetermined threshold value.

According to this configuration, the amount and type of cells to be replenished can be more appropriately calculated, so that cell therapy for myocardial necrosis can be appropriately supported.

### Sixth Embodiment

In the treatment support according to each of the embodiments described above, the amount of cells for the cell therapy may be further calculated on the basis of the survival rate of cultured cells introduced into a target site during culturing.

In the acquisition function 111, the processing circuitry 11 acquires information on the survival rate of cells to be provided for the cell therapy in a cell culturing phase. The processing circuitry 11 may acquire the survival rate from an external device of the support device 10, such as a culture device (not illustrated) or a management device that manages the culture, or may acquire the viability rate on the basis of the output of the input interface 17 in response to an operator's operation input.

In the determination function 115, the processing circuitry 11 corrects the amount of cells calculated on the basis of the cause information according to the survival rate in the culturing phase. For example, as the survival rate decreases, the processing circuitry 11 increases the amount of cells calculated on the basis of the cause information.

For example, the processing circuitry 11 corrects the acquired rate by using a relational formula or a table showing the relationship between a survival rate and a correction ratio determined in advance and stored in the storage circuitry 13 or the like. The relational formula or the table may indicate the relationship between the survival rate and the amount of correction. The relational formula or the table can be prepared for each cell type.

According to this configuration, the amount of cells to be replenished can be more appropriately calculated, so that cell therapy for myocardial necrosis can be appropriately supported.

### Seventh Embodiment

In the treatment support according to each of the embodiments described above, the display order of the therapeutic techniques that contribute to the cell therapy can be changed according to information on a patient's surgery.

In the acquisition function 111, the processing circuitry 11 acquires medical treatment data including information on the patient's surgery. The information on the patient's surgery includes information indicating whether the patient's surgery is acceptable. The acceptability of the patient's surgery is, for example, the acceptability of a thoracotomy surgery in the case of surgery for myocardial damage. The information indicating whether the patient's surgery is acceptable may include, for example, information indicating the patient's physical strength and acceptability of general anesthesia. The information indicating whether the patient's surgery is acceptable may be, for example, information indicating patient's desire such as refusal of surgery.

The display control function 117 ranks therapeutic techniques on the basis of information on the patient's surgery included in the medical treatment data acquired by the acquisition function 111, and displays the therapeutic techniques that contribute to the cell therapy. For example, when the information on the patient's surgery suggests that a patient is not able to tolerate the surgery, such as the patient's physical weakness or inability to undergo general anesthesia, the display control function 117 gives a higher ranking to a minimally invasive mesenchymal stem cell transplantation treatment technique. For example, when the information on the patient's surgery indicates the patient's desire such as refusal of surgery, the display control function 117 gives a higher ranking to a therapeutic technique that introduces cells by intravenous infusion.

In this way, the treatment support according to the present embodiment changes the display order of the therapeutic techniques that contribute to the cell therapy according to the information on the patient's surgery. According to this configuration, an operator such as a doctor can easily ascertain treatment plans that meet the patient's condition and desire.

### Application Example

An example of the flow of the treatment support according to the embodiment described above is described below.

A patient being treated for gastric cancer has presented with a complaint of suspected heart failure. This complaint is an example of a related chief complaint (supplementary information).

The patient was receiving anticancer drug therapy and received multiple courses of doxorubicin. The administration of the anticancer drug is, for example, the administration of a designated drug related to the myocardium that is registered in the medical treatment data as medication history information, and is an example of information on the administration of a drug to a patient.

Myocardial necrosis was strongly suspected due to LVEF of 50% on echocardiography and troponin level of 100 ng/mL on a blood test. These test results are examples of supplementary information.

The above supplementary information prompts the workflow in FIG. 3, and image diagnosis is performed on the basis of the information on the administration of the drug to the patient.

A cardiac contrast-enhanced MRI scan was performed, and necrosis of approximately 1% (equivalent to about 2 g) was suspected in the myocardium of the left ventricle. The suspected necrosis of the myocardium of the left ventricle is an example of identification of necrotic structures. The suspected necrosis of about 1% (equivalent to about 2 g) is an example of the size of a necrotic area (necrotic rate) at a specific time point.

Based on information on the total dosage 500 mg/m² of doxorubicin and information on the biological half-life 90 hours of doxorubicin, drug disappearance from the starting point of calculation (specific time point) was estimated to be after 900 hours (time point of disappearance), and the left ventricular myocardial necrosis was calculated to advance by approximately 10% (equivalent to 20 g). This calculation corresponds to the calculation of the amount of necrosis in the myocardium at the time point of disappearance when the occurrence of damage caused by the anticancer drug converges.

Cell types to be replenished at the time point of disappearance, 900 hours after the specific time point, were cardiomyocytes and vascular endothelial cells, and the amount of cells was estimated to be 1×10⁸. The cell types and the amount of cells to be replenished are the cell types and the amount of cells to be provided for the cell therapy.

Since the patient is not strong enough to undergo the thoracotomy surgery, mesenchymal stem cell transplantation, myocardial induction, and cell transplantation (iPS-derived myocardial differentiated cells) were listed in order.

In this way, the treatment support according to the embodiment can appropriately support cell therapy for site damage.

The term “processor” used in the above description means, for example, circuitry such as a CPU, a GPU, an ASIC, or a programmable logic device (PLD). The PLD includes a simple programmable logic device (SPLD), a complex programmable logic device (CPLD), and a field programmable gate array (FPGA). The processor implements functions by reading and executing computer programs stored in storage circuitry. The storage circuitry in which the computer programs are stored is a non-transitory computer readable recording medium. Instead of storing the computer programs in the storage circuitry, the computer programs may be directly incorporated in the circuitry of the processor. In this case, the processor implements the functions by reading and executing the computer programs incorporated in the circuitry. Instead of executing the computer programs, functions corresponding to the computer programs may be implemented by a combination of logic circuitry. Each processor of the present embodiments is not limited to being configured as single piece of circuitry for each processor, and one processor may be configured by combining a plurality of pieces of independent circuitry to implement the functions thereof. The plurality of components in FIG. 2 may be integrated into one processor to implement the functions thereof.

According to at least one of the embodiments described above, cell therapy for site damage can be appropriately supported.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

With respect to the above embodiments, the following supplementary notes are disclosed as an aspect and selective features of the invention.

Supplementary Note 1

A medical information processing device including processing circuitry configured to
acquire information on a cause of damage to a site of a patient,
specify an area where the damage is present in a medical image including the site, and
determine an amount of cells for cell therapy for the damage to the site on the basis of the acquired information on the cause and the specified area where the damage is present.

Supplementary Note 2

The processing circuitry may identify a tissue structure of the area where the damage is present.

The processing circuitry may determine a cell type for the cell therapy on the basis of the identified tissue structure.

Supplementary Note 3

The site may include a myocardium.

Supplementary Note 4

The information on the cause may include information on administration of a medicine to the patient.

Supplementary Note 5

The information on the administration of the medicine to the patient may include information on a type of the medicine.

Supplementary Note 6

The information on the administration of the medicine to the patient may include information on a total dosage of the medicine at a specific time point when the area where the damage is present is specified.

On the basis of the total dosage at the specific time point and a size of the specified area where the damage is present, the processing circuitry may predict an area where damage is present at a time point of disappearance when an occurrence of damage caused by the medicine converges, and on the basis of the size of the predicted area where the damage is present, the processing circuitry may determine the amount of cells for the cell therapy.

Supplementary Note 7

The processing circuitry may acquire supplementary information on a blood test for the patient.

The processing circuitry may specify the area where the damage is present when a marker increasing in blood with an occurrence of the damage to the site indicated by the supplementary information is equal to or greater than a predetermined threshold value.

The processing circuitry may specify the area where the damage is present when a marker increasing in blood with an occurrence of the damage to the site indicated by the supplementary information is positive.

Supplementary Note 8

The processing circuitry may acquire supplementary information on an interview to the patient.

The processing circuitry may specify the area where the damage is present when the supplementary information includes a chief complaint of the patient suggestive of the damage to the site.

Supplementary Note 9

The processing circuitry may acquire supplementary information on radiation therapy for the patient.

The processing circuitry may specify the area where the damage is present when a plan or a history of dose to the site indicated by the supplementary information satisfies a predetermined condition.

Supplementary Note 10

The information on the cause may include information on radiation therapy for the patient.

On the basis of a plan or a history of dose to the site in the radiation therapy, the processing circuitry may correct the amount of cells for the cell therapy.

Supplementary Note 11

The information on the cause may include information on radiation therapy for the patient.

On the basis of a plan or a history of dose to the site in the radiation therapy and a size of the specified area where the damage is present, the processing circuitry may predict an area where damage is present at a time point after radiation is applied to the site according to the plan or the history of the dose, and determine the amount of cells for the cell therapy on the basis of a size of the predicted area where the damage is present.

Supplementary Note 12

The processing circuitry may acquire information on a viability rate of cells to be provided for the cell therapy to the site.

The processing circuitry may correct the amount of cells for the cell therapy according to the viability rate.

Supplementary Note 13

The processing circuitry may acquire information on a survival rate of cells to be provided for the cell therapy to the site in a culturing phase.

The processing circuitry may correct the amount of cells for the cell therapy according to the survival rate in the culturing phase.

Supplementary Note 14

The processing circuitry may display, on a display, a list of therapeutic techniques that contribute to the cell therapy.

The processing circuitry may acquire information on surgery of the patient.

The processing circuitry may determine a display order of the therapeutic techniques in the list on the basis of the information on the surgery of the patient and a degree of invasiveness of the therapeutic techniques.

Supplementary Note 15

A medical information processing method including:
acquiring information on a cause of damage to a site of a patient;
specifying an area where the damage is present in a medical image including the site; and
determining an amount of cells for cell therapy for the damage to the site on the basis of the acquired information on the cause and the specified area where the damage is present.

Supplementary Note 16

A computer program product comprising instructions that cause a computer to execute:
acquiring information on a cause of damage to a site of a patient;
specifying an area where the damage is present in a medical image including the site; and
determining an amount of cells for cell therapy for the damage to the site on the basis of the acquired information on the cause and the specified area where the damage is present.

Supplementary Note 17

A computer program product executed by a computer and storing the computer program according to supplementary note 16.

What is claimed is:

1. A medical information processing device comprising processing circuitry configured to
acquire information on a cause of damage to a site of a patient,
specify an area where the damage is present in a medical image including the site, and
determine an amount of cells for cell therapy for the damage to the site on a basis of the acquired information on the cause and the specified area where the damage is present.

2. The medical information processing device according to claim 1, wherein the processing circuitry
identifies a tissue structure of the area where the damage is present, and
determines a cell type for the cell therapy on a basis of the identified tissue structure.

3. The medical information processing device according to claim 1, wherein the site includes a myocardium.

4. The medical information processing device according to claim 1, wherein the information on the cause includes information on administration of a medicine to the patient.

5. The medical information processing device according to claim 4, wherein the information on the administration of the medicine to the patient includes information on a type of the medicine.

6. The medical information processing device according to claim 4, wherein the information on the administration of the medicine to the patient includes information on a total dosage of the medicine at a specific time point when the area where the damage is present is specified, and
on a basis of the total dosage at the specific time point and a size of the specified area where the damage is present, the processing circuitry predicts an area where damage is present at a time point of disappearance when an occurrence of damage caused by the medicine converges, and on a basis of the size of the predicted area where the damage is present, the processing circuitry determines the amount of cells for the cell therapy.

7. The medical information processing device according to claim 1, wherein the processing circuitry
acquires supplementary information on a blood test for the patient, and
specifies the area where the damage is present when a marker increasing in blood with an occurrence of the damage to the site indicated by the supplementary information is positive.

8. The medical information processing device according to claim 1, wherein the processing circuitry acquires supplementary information on an interview to the patient, and specifies the area where the damage is present when the supplementary information includes a chief complaint of the patient suggestive of the damage to the site.

9. The medical information processing device according to claim 1, wherein the processing circuitry
acquires supplementary information on radiation therapy for the patient, and
specifies the area where the damage is present when a plan or a history of dose to the site indicated by the supplementary information satisfies a predetermined condition.

10. The medical information processing device according to claim 1, wherein the information on the cause includes information on radiation therapy for the patient, and
on a basis of a plan or a history of dose to the site in the radiation therapy, the processing circuitry corrects the amount of cells for the cell therapy.

11. The medical information processing device according to claim 1, wherein the information on the cause includes information on radiation therapy for the patient, and
on a basis of a plan or a history of dose to the site in the radiation therapy and a size of the specified area where the damage is present, the processing circuitry predicts an area where damage is present at a time point after radiation is applied to the site according to the plan or the history of the dose, and determines the amount of cells for the cell therapy on a basis of a size of the predicted area where the damage is present.

12. The medical information processing device according to claim 1, wherein the processing circuitry
acquires information on a viability rate of cells to be provided for the cell therapy to the site, and
corrects the amount of cells for the cell therapy according to the viability rate.

13. The medical information processing device according to claim 1, wherein the processing circuitry
acquires information on a survival rate of cells to be provided for the cell therapy to the site in a culturing phase, and
corrects the amount of cells for the cell therapy according to the survival rate in the culturing phase.

14. The medical information processing device according to claim 1, wherein the processing circuitry
displays, on a display, a list of therapeutic techniques that contribute to the cell therapy,
acquires information on surgery of the patient, and
determines a display order of the therapeutic techniques in the list on a basis of the information on the surgery of the patient and a degree of invasiveness of the therapeutic techniques.

15. A medical information processing method comprising:
acquiring information on a cause of damage to a site of a patient;
specifying an area where the damage is present in a medical image including the site; and
determining an amount of cells for cell therapy for the damage to the site on a basis of the acquired information on the cause and the specified area where the damage is present.

16. A computer program product comprising instructions that cause a computer to execute:
acquiring information on a cause of damage to a site of a patient;
specifying an area where the damage is present in a medical image including the site; and
determining an amount of cells for cell therapy for the damage to the site on a basis of the acquired information on the cause and the specified area where the damage is present.
